(19) United States
(12) Patent Application Publication
(10) Pub. No.: US 2025/0268754 A1
(43) Pub. Date: Aug. 28, 2025

(54) DYNAMIC SURGICAL FEEDBACK FOR EYE TISSUE

(71) Applicant: Alcon Inc., Fribourg (CH)

(72) Inventor: Paul R. Hallen, Colleyville, TX (US)

(21) Appl. No.: 19/046,622

(22) Filed: Feb. 6, 2025

Related U.S. Application Data
(60) Provisional application No. 63/556,493, filed on Feb. 22, 2024.

(51) Int. Cl. A61F 9/008; A61F 9/007

(52) U.S. Cl. CPC A61F 9/008; A61F 9/007; A61F 2009/00844; A61F 2009/00863; A61F 2009/00872

(57) ABSTRACT

In some embodiments, an ophthalmic surgical feedback system includes a laser device configured to emit light to a location on eye tissue of a patient and generate biomechanics information for the location based on the light. The ophthalmic surgical feedback system also includes a memory having executable instructions and a processor in communication with the laser device and the memory. The processor is configured to execute the instructions to determine strength of the eye tissue at the location based on the biomechanics information. The processor is further configured to execute the instructions to generate feedback related to utilization of a surgical instrument at the location based on the determined strength.

### INTRODUCTION

Various surgical tools, such as micro forceps, may be relied upon by surgeons to perform ophthalmic procedures. Use of such tools may occasionally result in adverse events, such as tissue tearing.

### SUMMARY

The present disclosure relates to surgical systems and methods, and more particularly, to systems and methods for dynamic surgical feedback related to eye tissue.

In some embodiments, one general aspect includes an ophthalmic surgical feedback system. The ophthalmic surgical feedback system includes a laser device configured to emit light to a location on eye tissue of a patient and generate biomechanics information for the location based on the light. The ophthalmic surgical feedback system also includes a memory having executable instructions and a processor in communication with the laser device and the memory. The processor is configured to execute the instructions to determine strength of the eye tissue at the location based on the biomechanics information. The processor is further configured to execute the instructions to generate feedback related to utilization of a surgical instrument at the location based on the determined strength.

In some embodiments, another general aspect includes a method for ophthalmic surgical feedback. The method includes emitting light from a laser device to a location on eye tissue of a patient. The method also includes generating biomechanics information for the location based on the light. The method also includes determining strength of the eye tissue at the location based on the biomechanics information. The method also includes generating feedback related to utilization of a surgical instrument at the location based on the determined strength.

### BRIEF DESCRIPTION OF THE DRAWINGS

So that the manner in which the above-recited features of the present disclosure can be understood in detail, a more particular description of the disclosure, briefly summarized above, may be had by reference to embodiments, some of which are illustrated in the appended drawings. It is to be noted, however, that the appended drawings illustrate only exemplary embodiments and are therefore not to be considered limiting of its scope, and may admit to other equally effective embodiments.

FIG. 1A illustrates an example configuration of an ophthalmic surgical feedback system (OSFS), according to certain embodiments of the present disclosure.

FIG. 1B illustrates another example configuration of an OSFS, according to certain embodiments of the present disclosure.

FIG. 2 is a block diagram of various components of the OSFS of FIGS. 1A-B, according to certain embodiments of the present disclosure.

FIG. 3 is a block diagram showing an example of an imaging device, according to certain embodiments of the present disclosure.

FIG. 4 illustrates an example of a process for operating an OSFS, according to certain embodiments of the present disclosure.

FIG. 5 illustrates an example of an ophthalmic suite operable to utilize an OSFS during surgery, according to certain embodiments of the present disclosure.

FIG. 6 illustrates an example of a process for providing intraoperative feedback, according to certain embodiments of the present disclosure.

To facilitate understanding, identical reference numerals have been used, where possible, to designate identical elements that are common to the figures. It is contemplated that elements and features of one embodiment may be beneficially incorporated in other embodiments without further recitation.

### DETAILED DESCRIPTION

For the purposes of promoting an understanding of the principles of the disclosure, reference will now be made to the implementations illustrated in the drawings, and specific language will be used to describe the same. It will nevertheless be understood that no limitation of the scope of the disclosure is intended. Any alterations and further modifications to the described systems, devices, instruments, methods, and any further application of the principles of the present disclosure are fully contemplated as would normally occur to one skilled in the art to which the disclosure relates In particular, the features, components, and/or steps described with respect to one implementation may be combined with the features, components, and/or steps described with respect to other implementations of the disclosure. For simplicity, in some instances the same reference numbers are used throughout the drawings to refer to the same or like parts.

The number and complexity of surgical procedures, including ophthalmic procedures, continue to increase every day. Examples include vitreoretinal procedures, cataract surgeries, corneal transplants, glaucoma surgeries, LASIK (Laser-Assisted In Situ Keratomileusis) surgeries, refractive lens exchanges, trabeculectomies, keratotomy procedures, and keratoplasty surgeries, etc. Various surgical instruments, such as micro forceps, phacoemulsification handpieces and/or the like, may be utilized to perform the procedures. Utilization of such surgical instruments often carries risk that an applied force or power, for example, will exceed a tolerance (e.g., a density or elasticity limit) of applicable eye tissue. Exceeding the tolerance can cause adverse events such as corneal damage (e.g., tearing, such as in the case of keratoconus), retinal damage (e.g., tearing and/or detachment), damage to an internal limiting membrane (TLM) (e.g., shredding), damage to an epi-retinal membrane (ERM) pathology (e.g., tearing), damage to a capsular bag (e.g., rupturing), damage to a cataract lens, and/or the like. Since the tolerance is typically variable throughout different areas of the eye tissue, it is technically challenging to appropriately control the surgical instrument in consideration of such tolerance, while also carrying out the procedure in an efficacious manner.

The present disclosure describes examples of systems and methods for dynamic, location-based feedback related to utilization of surgical instruments on eye tissue. In various embodiments, an ophthalmic surgical feedback system (OSFS) can use imaging technology, such as Brillouin spectroscopy, to generate biomechanics information for different locations on eye tissue. The biomechanics information can include, for example, one or more biomechanical properties of the eye tissue. A biomechanical property may be represented by an elastomechanical property or a viscoelastic property or a combination thereof and may be related to stiffness of the eye tissue at a given location. In various embodiments, a strength (e.g., tolerance) of the eye tissue at the different locations can be determined based on the biomechanics information.

In certain embodiments, the OSFS can generate feedback related to utilization of a surgical instrument, such as micro forceps or phacoemulsification handpieces (or other ophthalmic handpieces), based on the determined strength of the eye tissue at the different locations. In some embodiments, the feedback can include, for example, establishment of a value of one or more surgical parameters. The surgical parameters can vary with a type of the surgical instrument and can include, for example, instrument size, flow rate, force per unit area, speed of movement, power output, and/or the like. In addition, or alternatively, the feedback can include, for example, indications of how the strength of the eye tissue varies across the different locations (e.g., via a heat map). In addition, or alternatively, the feedback can include, for example, automated advice (e.g., selection of a particular location for utilization of the surgical instrument, a recommendation to proceed carefully due to relatively lower strength in a given area, etc.). Examples will be described relative to the Drawings.

For illustrative purposes, the present disclosure periodically describes various examples in relation to corneas. However, it should be appreciated that similar principles are applicable to measuring properties for other portions of the eye and/or other tissue.

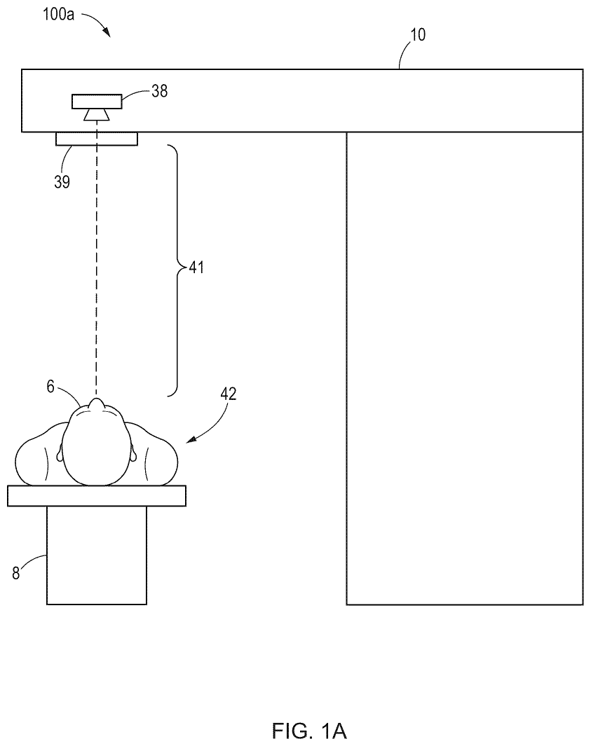

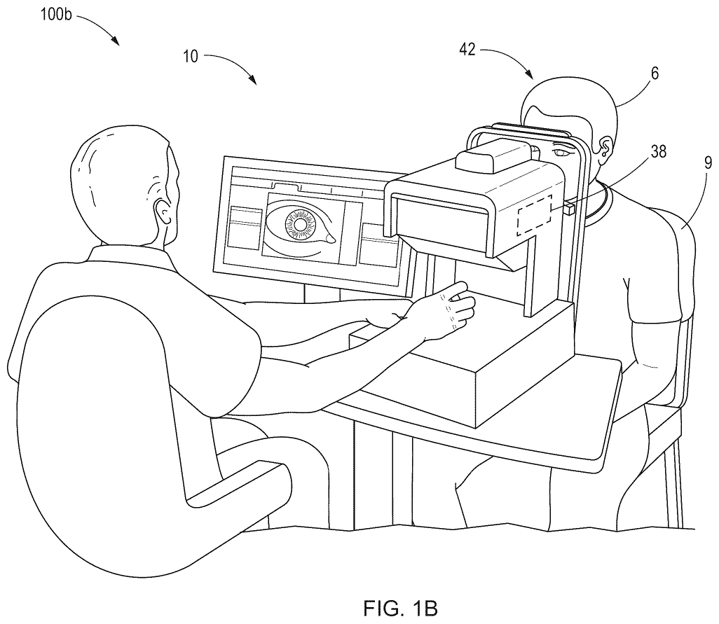

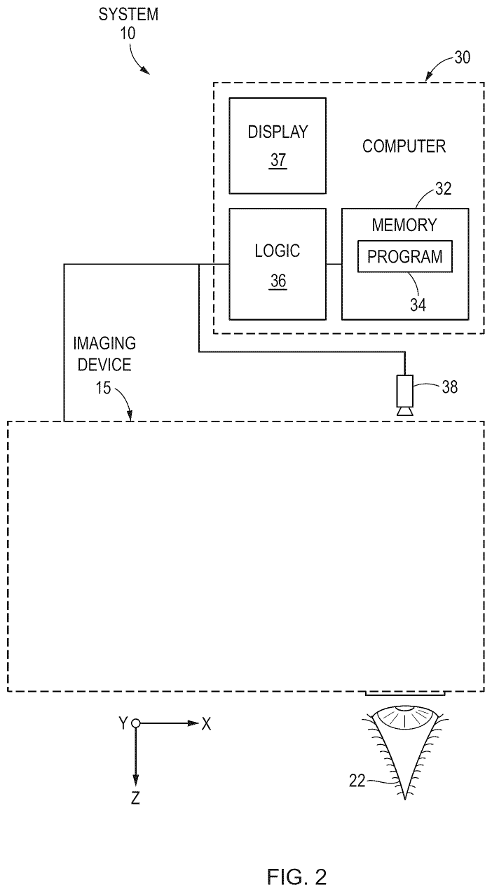

FIGS. 1A, 1, and 2 illustrate an example of an OSFS 10 according to certain embodiments. The OSFS 10 may be used for different types of diagnostic and treatment procedures. For example, the OSFS 10 may be used pre-operatively, intraoperatively, and/or the like.

FIG. 1A shows a configuration 100A of the OSFS 10. In particular, FIG. 1A illustrates a head 6 of a patient 42 lying on a bed 8. In the illustrated example, the OSFS 10 includes a camera 38 and a part 39 where multiple imaging light beams can exit the OSFS 10 and travel through an area 41 towards the patient 42.

FIG. 1B shows a configuration 100B of the OSFS 10. In the configuration 100B, the OSFS 10 is configured as a desktop imaging system in which the patient 42 sits in a chair 9.

With reference to FIG. 2, the OSFS 10 includes an imaging device 15, the camera 38, and a control computer 30, coupled as shown. The computer 30 includes logic 36, a memory 32 (which stores a computer program 34), and a display 37, coupled as shown. For ease of explanation, the following xyz-coordinate system is used: The z-direction is defined by the propagation direction of the imaging light beams, and the xy-plane is orthogonal to the propagation direction. Other suitable xyz-coordinate systems may be used.

The camera 38 can continuously capture one or more images of the patient 42. For example, the camera 38 can be focused on the eye 22. Examples of the camera 38 include a video, interferometry, thermal imaging, ultrasound, and eye-tracking cameras. The camera 38 delivers image data, which represent recorded images of the eye 22, to the computer 30. In some embodiments, the camera 38 can be an integral part of the imaging device 15, rather than separate as illustrated in FIG. 2.

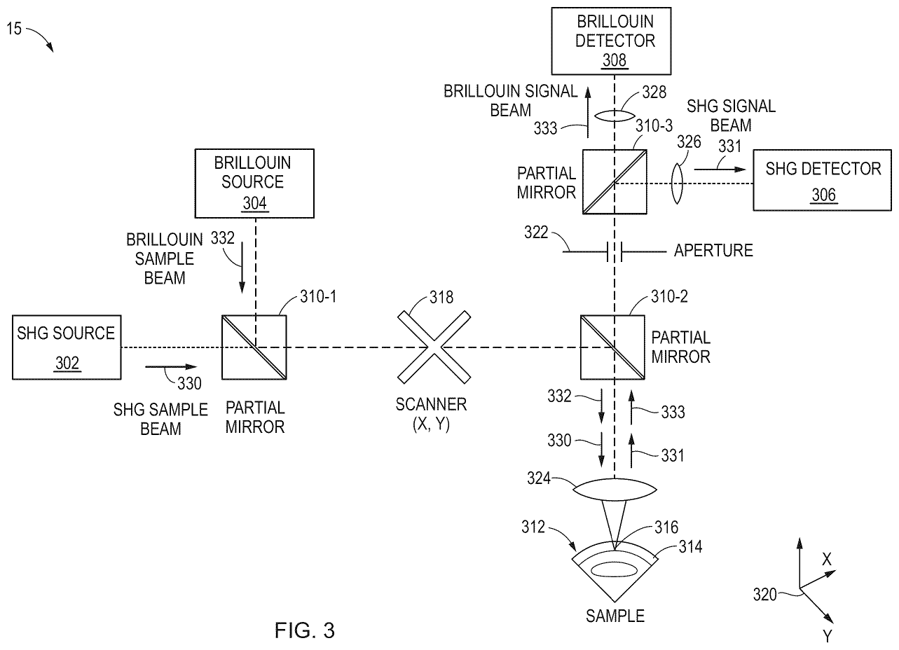

The imaging device 15 can generate and emit imaging light to tissue of an eye 22 of the patient 42. For example, the imaging light may be guided to a suitable location, for example, on a corneal or retinal surface of the eye 22. The imaging device 15 can generate biomechanics information for the location based on the light. In certain embodiments, the imaging device 15 can use, for example, Brillouin spectroscopy. An example of the imaging device 15 will be described in greater detail relative to FIG. 3.

For example, the cornea, or individual structural portions of the cornea, may be characterized as a linear-elastic, homogeneous or isotropic material. The biostructure of the cornea includes the conical epithelium, Bowman's layer (also known as the anterior limiting membrane), the corneal stroma (also known as substantia propria), Dua's Layer, Descemet's membrane (also known as posterior limiting membrane) and the corneal endothelium. Biomechanical properties may be characterized using different moduli. Examples are described below.

Stress may be defined as a restoring force in a material caused by a deformation divided by an area over which the restoring force is applied. Strain may be defined as a ratio of a change in a mechanical dimension of a material caused by stress with respect to an original state of the material.

A longitudinal modulus M (also known as a P-wave modulus or a constrained modulus) is used to describe isotropic homogeneous materials. The longitudinal modulus M is defined as a ratio of axial stress to axial strain in a uniaxial strain state where all other non-axial strains are zero, a state also referred to as zero lateral strain.

Young's modulus E (also referred to simply as an elastic modulus) is used to describe tensile elasticity. Tensile elasticity of a material is an axial deformation response when opposing forces are applied along an axis. Young's modulus E is defined as a ratio of tensile stress to tensile strain.

Lame's first parameter λ_{Lamé} (Greek: lambda-Lamé) may also be used to describe tensile elasticity.

A shear modulus G (also known as modulus of rigidity, p, Greek: mu, or Lame's second parameter) may be used to describe a shear deformation response of a material at constant volume when opposing forces are applied. The shear modulus G is defined as shear stress over shear strain and may be used to derive a viscosity of the material.

A bulk modulus K may be used to describe a volumetric elasticity or an isotropic deformation response of a material to an isotropic force, such as gas pressure. The bulk modulus K may be defined as volumetric stress over volumetric strain or as the inverse of compressibility κ (Greek: kappa). The bulk modulus K is an extension of Young's modulus E to three dimensions.

Poisson's ratio v (Greek: nu, also known as Poisson's number) may be used to describe a deformation response of a material, when compressed along a first axis, to expand in a second axis and a third axis both perpendicular to the first axis. Poisson's ratio v is defined as a negative ratio of transverse strain to axial strain or as a fraction of expansion divided by a fraction of compression.

For a homogeneous isotropic linear elastic material, certain equations are used to describe relationships among the various moduli described above. For example, the bulk modulus K, Young's modulus E, and the shear modulus G are related to Poisson's ratio v, as given in Equation 1.

$$v = \frac{E}{2 G} - 1 = \frac{3 K - E}{6 K} = \frac{3 K - 2 G}{6 K + 2 G} \qquad Equation 1$$

Also, the bulk modulus K, the shear modulus G, and the longitudinal modulus Mare related as given in Equation 2.

$$M = K + \frac{4 G}{3} \qquad Equation 2$$

In various embodiments, the imaging device 15 may employ Brillouin scattering to measure a biomechanical property of eye tissue. In Brillouin scattering, an acoustic wave, also referred to as a phonon, may indicate position dependent mass density variations inside a material. Because of localized compressions resulting from the mass density variation, an optical density of the material, also known as an index of refraction, may vary locally. The local variations in optical density n may result in a spatially periodic optical density variation, which behaves as a diffraction element for coherent light incident on the material. Brillouin scattering occurs when the coherent light interacts with such a diffraction element by being deflected or reflected from the material. Because the phonon is traveling within the material at a given velocity, light deflected or reflected from the phonon is subjected to a Doppler shift in frequency (or wavelength). In other words, the Brillouin scattered photons will have a different energy than the incident photons due to an inelastic scattering process. The change in the photon energy may be expressed as a change in frequency f (or wavelength λ), which are given in Equation 3.

$$f λ = \frac{c}{n} \qquad Equation 3$$

In Equation 3, c is the speed of light in vacuum and n is the optical density of the undisturbed material. The Brillouin scattering results in a frequency shift f_{B} (or a wavelength shift λ_{B}) that may be positive or negative with respect to the frequency f (or the wavelength λ) of the incident photon. Consequently, an inelastically Brillouin-scattered photon will have possible frequencies given by f±f_{B} (or possible wavelengths given by λ±λ_{B}). A spectrum of the Brillouin scattered light will include elastically deflected or reflected light, forming a Rayleigh peak at frequency f (or wavelength λ), along with the inelastically Brillouin-scattered light, forming at least one additional side peak (also referred to as a side band). When the side peak results from a scattered photon with higher energy than the incident photon, a Stokes peak at f+f_{B} (or at λ−λ_{B}) may be observed. When the side peak results from a scattered photon with lower energy than the incident photon, an anti-Stokes peak at f−f_{B} (or at λ+λ_{B}) may be observed.

In general, Brillouin-scattered photons will change propagation direction, such that the frequency shift f_{B} of the Brillouin-scattered light depends on a scattering angle θ between the incident photon and the Brillouin-scattered photon, as given in Equation 4.

$${f}_{B} = \pm \frac{2 n V}{λ} cos ( ∅ / 2 ) \qquad Equation 4$$

In Equation 4, n is the optical density of the undisturbed material, V is the velocity of the phonon in the material, λ is the wavelength of the incident photon in vacuum, and θ is the scattering angle. Per definition, the propagation direction of the incident photon is anti-parallel to the propagation direction of the Brillouin-scattered photon when θ is zero such that the incident photon is normal to a surface of the material. In Equation 4, the positive (+) result corresponds to the anti-Stokes Brillouin peak, while the negative result (−) corresponds to the Stokes Brillouin peak. For wavelengths |λ_{B}|<<λ, Equation 5 describes the relationship between f_{B} and λ_{B}.

$$\vert {f}_{B} \vert \approx \frac{\vert {λ}_{B} \vert}{{λ}^{2}} \qquad Equation 5$$

Because the frequency shift f_{B} depends on the scattering angle θ, each scattering angle θ is associated with a specific frequency shift f_{B}. A maximum or minimum value of the frequency shift is obtained by setting 0=0° in Equation 3, resulting in Equation 6, which corresponds to a normal incident light beam on the Brillouin scattering material.

$${f}_{B} = \pm \frac{2 n V}{λ} \qquad Equation 6$$

In the special case of 0=0°, the frequency shift f_{B} may be referred to as a longitudinal Brillouin shift.

By spectroscopically analyzing the Brillouin scattered light beam, certain biomechanical properties of the scattering material may be determined. For example, a complex valued longitudinal modulus M depends on the velocity of the phonon V as given by Equation 7.

$$M = {M}_{1} + i {M}_{2} + = ρ {V}^{2} + i ρ {V}^{2} [ \frac{Δ {f}_{B}}{{f}_{B}} ] \qquad Equation 7$$

In Equation 7, ρ is a mass density of the material in which the phonon propagates, and Δf_{B} is the line width of the Brillouin scattering side band.

The line width Δf_{B} corresponds to the reciprocal of a lifetime of the phonon and characterizes the attenuation of the phonon (sound wave) during propagation through the material. In one embodiment, the line width Δf_{B} may be measured as the full width at half maximum (FWHM) of the Stokes or anti-Stokes Brillouin peak. In other embodiments, another suitable definition of a spectral width that characterizes the frequency interval Δf_{B} may be used. For example, an amplitude of all spectral components may be assumed to be equal to or greater than a specified fraction of a spectral component having a maximum amplitude.

When the Brillouin scattered photon emerges in the anti-parallel direction to the incident photon, such as when θ=0°, the shear modulus G will be zero and the longitudinal modulus M will equal the bulk modulus, as is evident from Equation 2. In this case, the values M₁ and M₂ for the complex valued longitudinal modulus M will be respectively given by Equations 8 and 9.

$${M}_{1} = \frac{{λ}^{2} ρ}{4 {n}^{2}} {f}_{B}^{2} \qquad Equation 8$$

$${M}_{2} = \frac{{λ}^{2} ρ}{4 {n}^{2}} {f}_{B} Δ {f}_{B} \qquad Equation 9$$

In Equation 8, M₁ describes an elastomechanical property of the material, while in Equation 9, M₂ describes a viscoelastic property of the material. Accordingly, by measuring the frequency shift f_{B} of one of the side bands (either Stokes or anti-Stokes) of a Brillouin scattered light beam backscattered from a material (also referred to as a Brillouin signal beam) in response to an incident beam (also referred to as a Brillouin sample beam), an elastomechanical property of the material may be determined. Furthermore, by also measuring the line width Δf_{B} of the side band, a viscoelastic property of the material may be determined.

In addition to Brillouin scattering, second harmonic generation (SHG) signals from human cornea have been associated with the position and distribution of the fibrils in the cornea. SHG refers to second order nonlinear emission of photons at half-wavelength by a material in response to excitation at full-wavelength, as given by Equation 10.

$${λ}_{SHG} = {λ}_{1} / 2 \qquad Equation 10$$

In Equation 10, λ_{SHG} is the wavelength of the SHG signal in a SHG signal beam and λ₁ is the wavelength of the excitation beam (also referred to as a SHG sample beam).

The excitation for SHG signals from human cornea may be performed using a fs-fiber laser focused at a desired sampling location for precise spatial collection of SHG signals that can be used to generate images of conical biostructures. In some embodiments, the excitation area may be on the order of a few microns when collecting SHG signals from human cornea. Fibrils in the cornea are comprised of collagen, which is known to be a highly effective nonlinear SHG signal source. Furthermore, the nonlinear interaction of the excitation beam with corneal collagen fibrils is dependent on the position, orientation, density, and alignment of the collagen fibrils, which may result in the SHG signal providing significant insight into the biostructural condition of various corneal tissues.

For in vivo imaging, backward SHG signals (B-SHG) may be obtained from human cornea. The B-SHG signal beam may be emitted in a roughly anti-parallel direction to the incident SHG sample beam and may be detected by any suitable optical detection system. In some embodiments, a photomultiplier tube (PMT) may be used as an SHG detector for high sensitivity applications of imaging using B-SHG signals. In some embodiments, a multi-channel plate detector, which is similar to a PMT but provides further spatial resolution using a plurality of separate channels, may be used as the SHG detector. The SHG detector may be equipped with an optical filter to discriminate the λ_{SHG}wavelength from the measurement beam returning from the sampled material. When a PMT or similar photodetector is used, a photocathode material may be selected for a desired sensitivity to the λ_{SHG} wavelength. Furthermore, in some instances, polarization of the SHG sample beam may be employed for additional selectivity to particular emission modes of the sampled material. The polarization-sensitive emission modes may be related to morphological features of collagen fibrils when the sampled material is human corneal tissue. When the SHG sample beam is polarized, the SHG detector may also include a polarization filter to discriminate various polarization orientations in the SHG signal beam.

Still with reference to FIG. 2, the computer 30 controls components of the OSFS 10 in accordance with the computer program 34. The memory 32 stores information used by the computer 30. For example, memory 32 may store images of the eye 22, the imaging data (e.g., including the biomechanics information), feedback (as discussed further below), and/or other suitable information, and the computer 30 may access information from the memory 32. In various embodiments, the computer program 34 and its functionality can be directed by a user such as a medical professional.

More particularly, the computer 30 can instruct, or cause, the imaging device 15 to emit imaging light to different desired measurement locations on the eye 22 (e.g., the cornea or retina), and to generate the biomechanics information based on the imaging light. In some embodiments, the computer 30 can cause the biomechanics information to be generated in the foregoing fashion via a scan of a portion of the eye 22. The computer 30 can then receive, in real-time from the imaging device 15, imaging data resultant from the imaging light, including biomechanics information as described above and in more detail relative to FIG. 3. In various embodiments, the computer 30 can receive biomechanics information for multiple areas of the eye 22.

In various embodiments, the computer 30 can determine a strength of the eye tissue at each of the different locations based on the biomechanics information. In some cases, the strength can result from the computer 30 contextualizing the biomechanics information, for example, by applying one or more thresholds to particular biomechanical properties. In some embodiments, strength for each location may be a quantification based on a predetermined scale. For example, the scale may be 1 to N (e.g., 1 to 10 or 1 to 100), with formulas or rules defining how the strength is quantified in terms of a particular biomechanical property and/or combination of biomechanical properties.

In certain embodiments, the computer 30 can generate feedback related to utilization of one or more surgical instruments at each of the different locations based on the determined strength. In certain embodiments, the computer 30 can output the feedback it generates. For example, the computer 30 can record the feedback in the memory 32 or other storage. In addition, or alternatively, the computer 30 can present or display the feedback on the display 37. In general, the computer 30 can make the feedback available to a surgeon or other user in any suitable fashion including, for example, in an audio, visual, and/or haptic manner.

The feedback generated by the computer 30 can take various forms according to a configuration of the OSFS 10. For example, the feedback can be a summarization, such as a graphical summarization, of how the determined strength varies across the different locations on the eye tissue (e.g., a heat map). In addition, or alternatively, the feedback can be a value of a surgical parameter related to utilization of the surgical instrument, such as instrument size, flow rate, force per unit area, speed of movement, power output, combinations of the foregoing and/or the like. In such cases, the value can be established based on the strength at the different locations, for example, in a rule or formula-based manner.

In addition, or alternatively, the feedback can include advice relative to one or more of the different locations. For example, the feedback can include a recommendation to exercise caution with the surgical instrument at a given location because the strength is below a threshold. In another example, the feedback generated by the computer 30 can include advice about where to utilize the surgical instrument. According to this example, the computer 30 can select a location, from among the different locations, for utilization of the surgical instrument. The selection can be based on configurable criteria, such as the location where the eye tissue is deemed to be strongest.

In addition, or alternatively, the feedback can be a control parameter for a robotic surgical system. For example, a value of a surgical parameter as described above can be provided to the robotic surgical system as a command or configuration for utilization of the surgical instrument. In another example, a selected location as described above can be provided to the robotic surgical system as a command regarding where to utilize the surgical instrument.

In certain embodiments, determination of strength and/or generation of feedback can be at least partially based in machine learning (e.g., supervised learning). For example, the memory 32 can include models trained on datasets for a large set of patients. According to this example, the datasets on which the models are trained can include records detailing sets of features such as eye conditions, biomechanics information for specific eye-tissue locations, and information describing each patient (e.g., age, gender, and ethnicity). Each record can further include, or be labeled with, strength for particular locations and/or feedback for particular locations. Therefore, according to this example, the computer program 34 can use the patient's eye condition, biomechanics information, and/or available information describing the patient (e.g., age, gender, ethnicity, etc.) to predict or determine strength and/or feedback.

FIG. 3 is a block diagram showing the imaging device 15. As shown, imaging device 15 is used to analyze sample 312, which may represent a human eye such as eye 22 of FIG. 2, and in particular, to analyze a cornea 314 of the human eye. Also, in imaging device 15, coordinate system 320 defines an axial direction in Z and lateral directions in X and Y, which are relative to sample 312 such that SHG sample beam 330 and Brillouin sample beam 332 propagate towards sample 312 in the axial direction Z. Imaging device 15 accordingly enables simultaneous capture of both Brillouin signals and SHG signals from sample 312 using a measurement process that is spatially correlated. In this manner, imaging device 15 may enable improved analysis and measurement of certain physical properties of eye tissue in sample 312 in many diagnostic and clinical applications.

As shown, imaging device 15 includes SHG source 302 from which SHG sample beam 330 is generated. When SHG source 302 is a fs-fiber laser, the λ₁ wavelength may be 1030 nanometers (nm) in particular embodiments, and the λ_{SHG} wavelength may correspondingly be 515 nm. Imaging device 15 further includes Brillouin source 304 from which Brillouin sample beam 332 is generated. Brillouin source 304 may be any narrowband light source suitable for Brillouin scattering in eye tissue. In some embodiments, Brillouin source 304 is a single mode continuous wave laser having a wavelength of 532 nm and a line width of about 1 MHz (megahertz). SHG source 302 and Brillouin source 304 may be positioned to be confocal with respect to sample 312 at focus position 316, which may be adjusted using focusing lens 324.

In FIG. 3, SHG sample beam 330 and Brillouin sample beam 332 are combined into a single optical path at partial mirror 310-1. The combined beam of SHG sample beam 330 and Brillouin sample beam 332 may be spatially modulated in the X-Y plane using scanner 318, in order to scan various locations in sample 312. Scanner 318 may accordingly modulate focus position 316 in the X-Y plane to sample various locations in sample 312, such as different areas of interest in cornea 314. From scanner 318, the combined beam of SHG sample beam 330 and Brillouin sample beam 332 may propagate to sample 312 at partial mirror 310-2, via focusing lens 324. Focusing lens 324 may be adjustable in the Z axis using any suitable mechanism to vary a focus position 316 along the Z axis. Thus, in the embodiment shown in FIG. 3, SHG sample beam 330 and Brillouin sample beam 332 are propagated to sample 312 along a common optical path from partial mirror 310-1, which serves as a common optical start point for both SHG sample beam 330 and Brillouin sample beam 332.

From partial mirror 310-2 towards sample 312, the combined beam of SHG sample beam 330 and Brillouin sample beam 332 may propagate to sample 312 in a normal or substantially normal direction to a surface of sample 312. To the extent that the combined beam has a certain beam width, focusing lens 324 may bundle SHG sample beam 330 and Brillouin sample beam 332 to a desired sample area at focus position 316. Then, a combined beam of SHG signal beam 331 and Brillouin signal beam 333 may be scattered back from sample 312 towards partial mirror 310-2. It is noted that the sampling geometry depicted in imaging device 15 is exemplary and may be modified in different embodiments.

From partial mirror 310-2 towards partial mirror 310-3, the combined beam of SHG signal beam 331 and Brillouin signal beam 333 may propagate through aperture 322. Aperture 322 may be confocally arranged with respect to SHG signal beam 331 and Brillouin signal beam 333. Aperture 322 may be used to limit photons in SHG signal beam 331 and Brillouin signal beam 333 to a particular scan angle, for example, depending on scanner 318. Accordingly, aperture 322 may be mechanically adjustable depending on a scan angle used by scanner 318. In other embodiments, focusing lens 324 may be used to center or align SHG sample beam 330 and Brillouin sample beam 332, such that SHG signal beam 331 and Brillouin signal beam 333 are aligned with aperture 322, for example, when aperture 322 is fixed.

At partial mirror 310-3, SHG signal beam 331 may be directed to SHG detector 306 via detection lens 326, while Brillouin signal beam 333 may be directed to Brillouin detector 308 via detection lens 328. Both detection lenses 326 and 328 may be arranged confocally with respect to SHG signal beam 331 and Brillouin signal beam 333. SHG detector 306 may be any suitable detector for SHG signal beam 331, such as a PMT or a multi-channel plate detector, as described above.

Brillouin detector 308 may include a high-resolution spectrometer suitable for discriminating Rayleigh scattering from Brillouin scattering. Because the Rayleigh scattered beam may have a significantly greater amplitude than the Brillouin scattered beam and both scattered beams may be relatively close together spectrally, Brillouin detector 308 may have high spectral resolution and also high spectral contrast. In particular embodiments, Brillouin detector 308 may include a charge-coupled device (CCD) array as an optical sensor.

In operation of imaging device 15, the combined beam of SHG sample beam 330 and Brillouin sample beam 332 may be confocally propagated to focus position 316, which may be modulated in the X-Y plane using scanner 318. Focus position 316 may be modulated in Z using focusing lens 324. In this manner, various points, lines, areas, and volumes in sample 312 may be scanned and analyzed using imaging device 15.

At focus position 316, Brillouin signal beam 333 may be measured by Brillouin detector 308. Specifically, Brillouin detector 308 may measure the frequency shift f_{B} of one (or both) of the side bands (either Stokes or anti-Stokes) in Brillouin signal beam 333. Brillouin detector 308 may also measure the line width Δf_{B} of one or both of the side bands. With the measured frequency shift f_{B} and the measured line width Δf_{B}, an elastomechanical property and a viscoelastic property at focus position 316 may be determined, as explained above with respect to Equations 8 and 9.

Simultaneously and from the same focus position 316, SHG signal beam 331 may be measured by SHG detector 306. Specifically, SHG detector 306 may register a signal amplitude of the wavelength in SHG signal beam 331. In particular, SHG detector 306 may be sensitive to small amplitudes at the λ_{SHG} wavelength. Because the signal amplitude of the λ_{SHG} wavelength is indicative of a morphological structure of collagen fibrils at focus position 316, the signal amplitude registered by SHG detector 306 may be used to generate certain images of eye tissue in sample 312. The image information generated by SHG detector 306 in this manner may be precisely spatially correlated with the elastomechanical property and the viscoelastic property at focus position 316. The resulting data generated by imaging device 15 may provide a more complete understanding and analysis of a condition of sample 312 at focus position 316.

It is noted that, in various embodiments or arrangements of imaging device 15, different implementations, layouts and diversions of beams may be used. For example, certain portions of optical paths used in imaging device 15 may include optical fibers. In some embodiments, certain portions of optical paths used in imaging device 15 may include optical waveguides. Certain portions of optical paths used in imaging device 15 may represent optical paths within a medium, such as vacuum, free space, a gaseous environment, or the atmosphere. In given embodiments, a polarizing element may be used with at least one of SHG sample beam 330 and Brillouin sample beam 332, and a polarization filter may be used when detecting at least one of SHG signal beam 331 and Brillouin signal beam 333. In another arrangement, scanner 318 may be omitted and another scanning element, such as an objective, may be used. In particular embodiments, at least a portion of the optical components included with imaging device 15 may be miniaturized and combined into a compact unit having relatively small mass and external dimensions, such that the entire compact unit is held by an external scanning element and moved with respect to sample 312. Also, different orientations of coordinate system 320 may be used in certain embodiments of imaging device 15.

In various embodiments, imaging device 15 may be used to characterize or analyze intra-corneal layer biostructures, such as fibrils or microfibrils in human corneal stroma.

It is noted that imaging device 15 is not drawn to scale but is a schematic representation. Modifications, additions, or omissions may be made to imaging device 15 without departing from the scope of the disclosure. The components and elements of imaging device 15, as described herein, may be integrated or separated according to particular applications. Moreover, the operations of imaging device 15 may be performed by more, fewer, or other components.

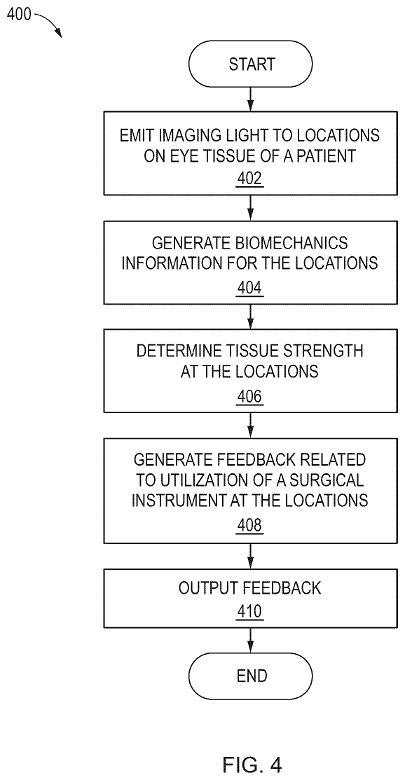

FIG. 4 illustrates an example of a process 400 for operating an OSFS. In certain embodiments, the process 400 can be implemented by any system that can process imaging data. Although any number of systems, in whole or in part, can implement the process 400, to simplify discussion, the process 400 will be described in relation to example components of the OSFS 10 of FIGS. 1A-B, 2 and 3.

At block 402, the imaging device 15 emits imaging light to different locations on eye tissue of a patient. At block 404, the imaging device 15 generates biomechanics information for the different locations on the eye tissue. In certain embodiments, the blocks 402 and 404 can involve the computer 30 causing, or instructing, the imaging device 15 to emit the imaging light and generate the biomechanics information, respectively, as described relative to FIGS. 1A-B, 2, and 3. In some embodiments, the blocks 402 and 404 can represent an execution of a scan of the eye tissue across the different locations.

At block 406, the computer 30 determines a strength of the eye tissue at the different locations based on the biomechanics information. In general, the block 406 can involve contextualizing the biomechanics information as described relative to FIG. 2. For example, the computer 30 can apply one or more thresholds to particular biomechanical properties to assess or identify strength. In other examples, strength for each location may be a quantification based on a predetermined scale (e.g., a 1 to N scale).

At block 408, the computer 30 generates feedback related to utilization of a surgical instrument at the different locations based on the determined strength. In various embodiments, the feedback can be generated in any of the ways described relative to FIG. 2. For example, the block 408 can involve establishing a value of one or more surgical parameters related to the utilization of the surgical instrument at each of the different locations. In other examples, the block 408 can include generating advice or recommendations. In still other examples, the block 408 can involve selecting one or more of the different locations for utilization of the surgical instrument.

At block 408, the computer 30 outputs the generated feedback. The output at the block 408 can involve recording the feedback in the memory 32 or other storage, presenting the feedback on a display such as the display 37, providing the feedback as a command to a robotic surgical system, combinations of the foregoing and/or the like. After block 408, the process 400 ends.

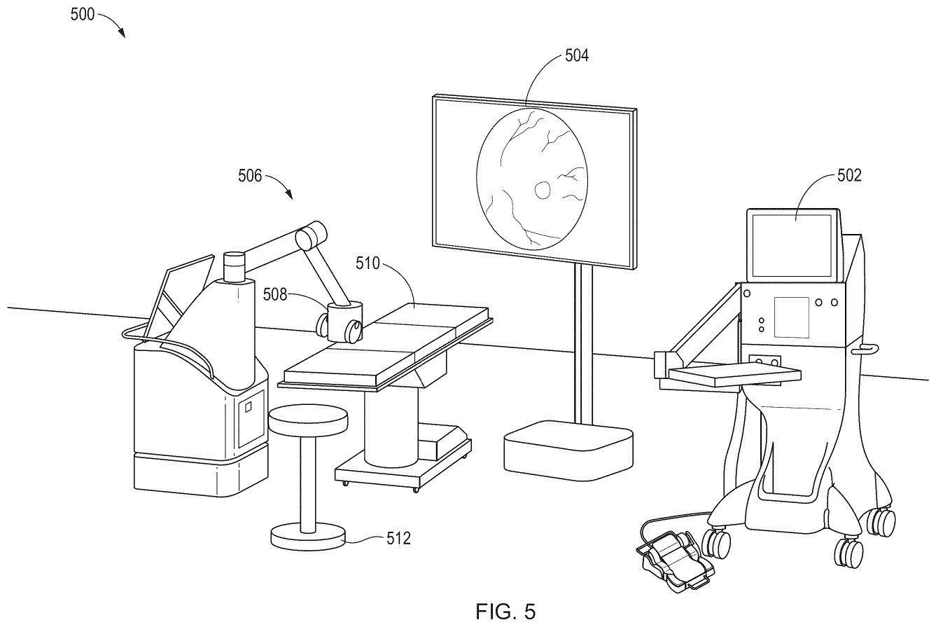

FIG. 5 illustrates an ophthalmic suite 500 operable to utilize an OSFS during surgery. The ophthalmic suite 500 includes a surgical console 502, a heads-up display 504, and a surgical camera system 506 that positions a surgical camera 508 over a patient table 510. The surgical camera 508 can be a High Dynamic Range (HDR) camera with a resolution, image depth, clarity and color contrast that enables a high quality, three-dimensional views of an eye as well as surgical consoles and heads-up displays for performing actions during ophthalmic procedures that use the HDR camera.

The surgical camera 508 can be communicatively coupled with the heads-up display 504 (e.g., via a wired connection, a wireless connection, etc.) and the heads-up display 504 can display a stereoscopic representation of the three-dimensional image providing a surgeon, staff, students, and other observers depth perception into the eye anatomy. The surgical camera 508 can also be used to increase magnification of the eye anatomy while maintaining a wide field of view. The stereoscopic representation of the three-dimensional image can be viewed on the heads-up display with stereoscopic glasses, as an autostereogram, using Fresnel lenses, etc. With the stereoscopic representation of the three-dimensional image displayed on the heads-up display 504, a surgeon can perform procedures on a patient's eye while in a comfortable position (e.g., sitting on a stool 512) without bending over a microscope eyepiece and straining his neck.

The surgical console 502 can be communicatively coupled with the heads-up display 504 and/or the surgical camera system 506. In some embodiments, the heads-up display 504 can receive information from the surgical console 502 and display the information on the heads-up display 504 along with the stereoscopic representation of the three-dimensional image. The surgical console 502 can also send signals to the heads-up display 504 for performing operations (e.g., starting and stopping video recording).

In addition, ophthalmic suite 500 can also include a surgical suite optimization engine containing one or more processors and memory for performing advanced operations. As explained in greater detail below, the surgical suite optimization engine can integrate a surgical suite and can perform a wide variety of actions to synergistically optimize a wide variety of surgical functions. For example, the surgical suite optimization engine can: enable gaze-tracking to navigate a display dashboard; perform digital signal processing for adjusting display settings, applying filters, increasing contrast, neutralizing particular wavelengths, identifying anatomy, etc.; display of a wide variety of images at various zoom depths, menus, other diagnostic images, surgical productivity applications, surgical schedules, live video teleconferencing, etc.; control robotic arms to move the heads-up display to optimize stereopsis or ensure centration of the surgical camera; control a diagnostic device to automatically focus on anatomy based on the procedural step; provide alerts and recommendations to surgical staff, modulate color effects during laser treatment; etc.

Also, surgical suite optimization engine can include a network interface that allows the surgical suite optimization engine to communicate with the surgical camera system 506, the surgical console 502, and other surgical systems. In some cases, the surgical suite optimization engine can serve to inter-network some or all of network-capable components in ophthalmic suite 500.

Inter-networking the surgical suite can allow for advancements in surgical practice and patient outcome. For example, the surgical console 502 can include a wide assortment of tools for performing various aspects of ophthalmic procedures and can include memory and one or more processors for controlling the tools as well as monitoring a wide variety of quantitative features of a surgical procedure. When the surgical console 502 is connected with the surgical suite optimization engine, the surgical console 502 sends information about the quantitative features of a surgical procedure (e.g., via one or more action codes) to the surgical suite optimization engine. In response, the surgical suite optimization engine can interpret the information from the surgical console 502 and perform actions that improve other areas of a surgical procedure or a surgical practice. More generally, the surgical suite optimization engine can receive, via the network interface, an assortment of information from any of the components in the ophthalmic suite 500 and, in response to the gathered information, perform actions that improve a wide variety of areas of a surgical procedure or a surgical practice, resulting in better patient outcomes.

The surgical console 502 can include, or be in communication with, an OSFS such as the OSFS 10 of FIGS. 1A-B and 2. In some embodiments, the surgical console 502 can perform any of the functionality described relative to FIGS. 1A-B and 2-4. For example, in some embodiments, the surgical console 502 can perform the process 400, or cause the process 400 to be performed. In some embodiments, the surgical console 502 may cause a process such as the process 400 to be repeated, for example, to intraoperatively refresh biomechanics information and/or feedback.

In various embodiments, the surgical console 502 can cause the heads-up display 504 to overlay a three-dimensional view of an eye with feedback of the type described above relative to FIGS. 1A-B and 2-4. In an example, the three-dimensional view can be overlaid with information indicating a relative strength at different locations on eye tissue of interest (e.g., corneal or retinal tissue as described previously). The relative strength can be indicated as a heat map in some cases. In other examples, the three-dimensional view can be overlaid with feedback indicating, for one or more particular locations on the eye tissue, advice or recommendations, one or more selected locations for utilization of a surgical instrument, and/or the like.

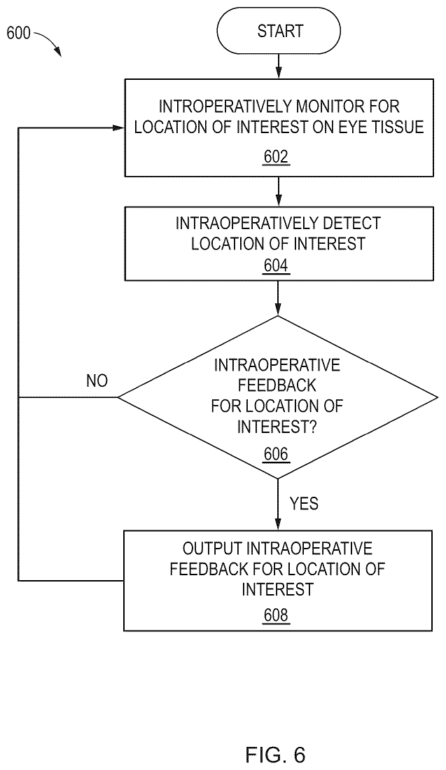

In other examples, the surgical console 502 can output timely, intraoperative feedback to a surgeon. For example, the surgical console 502 can detect a location of interest on the eye tissue (e.g., based on a current zoom level of the three-dimensional view or a manual indication) and provide feedback related to utilization of the surgical instrument at the location of interest, such as any of the feedback described previously. For example, the feedback can be displayed on the heads-up display 504 or otherwise reported to the user in any suitable fashion (e.g., in an audio, visual and/or haptic manner). In some embodiments, the surgical console 502 can detect the location by monitoring movement of the surgical instrument relative to the eye tissue. An example of providing intraoperative feedback will be described relative to FIG. 6.

FIG. 6 illustrates an example of a process 600 for providing intraoperative feedback. In various embodiments, the process 600 can be initiated manually or automatically, for example, at the beginning of an ophthalmic surgical procedure. In certain embodiments, the process 600 can be implemented by any system that can process imaging data. Although any number of systems, in whole or in part, can implement the process 600, to simplify discussion, the process 600 will be described in relation to example components of the surgical console 502 of FIG. 5.

At block 602, the surgical console 502 monitors for a location of interest on eye tissue. In various embodiments, the surgical console 502 can detect one or more locations of interest, for example, based on what is displayed at a current zoom level of a three-dimensional display as described relative to FIG. 5. In other examples, the surgical console 502 can detect one or more locations of interest based on a manual indication by a surgeon or other user. In still other examples, the surgical console 502 can detect one or more locations of interest by monitoring movement of a surgical instrument. Although the surgical console 502 may detect multiple locations at once in some implementations, for simplicity, the process 600 will be described relative to a single location.

At block 604, the surgical console 502 detects a location of interest responsive to the monitoring at the block 602. At decision block 606, the surgical console 502 determines whether there is intraoperative feedback to present relative to the location of interest. In general, the intraoperative feedback can include any of the example feedback described previously relative to FIGS. 1A-B and 2-5. In some embodiments, such as where the location of interest corresponds to one of the locations for which feedback was previously generated as described relative to FIGS. 1A-B and 2-5, the decision block 606 can involve retrieving the previously generated feedback and treating such feedback as the intraoperative feedback. In addition, or alternatively, the decision block 606 can include generating some or all of the intraoperative feedback for the location of interest in any of the ways described previously.

If there is no intraoperative feedback for the location of interest, the process 600 returns to the block 602, where the surgical console 502 continues to monitor for locations of interest as described previously. Otherwise, if it is determined, at the decision block 606, that there is intraoperative feedback for the location of interest, at block 608, the surgical console 502 outputs the intraoperative feedback for the location of interest, for example, as described relative to FIG. 5. After block 608, the process 600 returns to block 602, where the surgical console 502 monitors for a location of interest as described previously. In various embodiments, the process 600 can continue until terminated by a surgeon or other user, or until other suitable stop criteria is satisfied.

The above-disclosed subject matter is to be considered illustrative, and not restrictive, and the appended claims are intended to cover all such modifications, enhancements, and other embodiments which fall within the true spirit and scope of the present disclosure. Thus, to the maximum extent allowed by law, the scope of the present disclosure is to be determined by the broadest permissible interpretation of the following claims and their equivalents, and shall not be restricted or limited by the foregoing detailed description.

What is claimed is:

1. An ophthalmic surgical feedback system, comprising:
a laser device configured to emit light to at least one location on eye tissue of a patient and generate biomechanics information for the at least one location based on the light;
a memory comprising executable instructions; and
a processor in communication with the laser device and the memory and configured to execute the instructions to:
determine strength of the eye tissue at the at least one location based on the biomechanics information; and
generate feedback related to utilization of a surgical instrument at the at least one location based on the determined strength.

2. The ophthalmic surgical feedback system of claim 1, wherein the eye tissue comprises retinal tissue.

3. The ophthalmic surgical feedback system of claim 1, wherein the eye tissue comprises corneal tissue.

4. The ophthalmic surgical feedback system of claim 1, wherein the generation of the feedback comprises establishment of a value of a surgical parameter related to the utilization of the surgical instrument at the at least one location.

5. The ophthalmic surgical feedback system of claim 4, wherein the surgical parameter comprises at least one of instrument size, flow rate, force per unit area, speed of movement, or power output.

6. The ophthalmic surgical feedback system of claim 1, wherein the processor is further configured to execute the instructions to at least one of record or display data related to the generated feedback.

7. The ophthalmic surgical feedback system of claim 1, wherein the at least one location comprises a plurality of locations on the eye tissue of the patient.

8. The ophthalmic surgical feedback system of claim 7, wherein the generation of the feedback comprises selecting one or more locations of the plurality of locations for utilization of the surgical instrument.

9. The ophthalmic surgical feedback system of claim 1, wherein the processor is further configured to:
intraoperatively detect a location of interest related to utilization of the surgical instrument;
determine intraoperative feedback for the location of interest; and
output the intraoperative feedback.

10. The ophthalmic surgical feedback system of claim 1, wherein the determination of strength comprises a quantification based on a predetermined scale.

11. The ophthalmic surgical feedback system of claim 1, wherein the generation of the biomechanics information is based on Brillouin spectroscopy.

12. The ophthalmic surgical feedback system of claim 1, wherein the processor is further configured to provide at least a portion of the feedback as a command to a robotic surgical system, wherein the at least a portion of the feedback comprises at least one of a command or a configuration related to utilization of the surgical instrument.

13. The ophthalmic surgical feedback system of claim 1, wherein the surgical instrument comprises at least one of forceps or an ophthalmic handpiece.

14. The ophthalmic surgical feedback system of claim 1, further comprising a display, wherein the processor is further configured to execute the instructions to cause the display to:
display an image of an eye; and
overlay the image with at least a portion of the feedback.

15. A method for ophthalmic surgical feedback, the method comprising:
emitting light from a laser device to a location on eye tissue of a patient;
generating biomechanics information for the location based on the light;
determining strength of the eye tissue at the location based on the biomechanics information; and
generating feedback related to utilization of a surgical instrument at the location based on the determined strength.
